(19) United States
(12) Patent Application Publication
(10) Pub. No.: US 2013/0161320 A1
(43) Pub. Date: Jun. 27, 2013

(54) MODULAR DELIVERY SYSTEM

(71) Applicant: Reese K. MYERS, Columbia City, IN (US)

(72) Inventor: Reese K. MYERS, Columbia City, IN (US)

(21) Appl. No.: 13/337,599

(22) Filed: Dec. 27, 2011

(51) Int. Cl. A61B 19/02; B65B 17/00; B65D 25/28; B65D 21/024; B65D 43/00

(52) U.S. Cl. 220/23.4; 53/473

(57) ABSTRACT

A method of packaging sterilized medical equipment, including providing a plurality of first containers each having a first size; placing at least one sterilized medical device in a first container of the plurality; providing a plurality of second containers each having a second size different from the first size; placing at least one sterilized medical device in a second container of the plurality; and releasably engaging the first container to the second container such that the engaged first and second containers define a substantially smooth exterior profile, where a size of the exterior profile conforms to an industry standard size.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to the field of containers and cases for holding, organizing, and sterilizing surgical instruments, implants and devices.

### BACKGROUND OF THE INVENTION

Surgical procedures are typically performed using collections or sets of pre-selected surgical instruments useful in a given surgical procedure. Typical tools or instruments include scalpels, hemostats, needles or probes, varying prostheses, and/or glassware such as Ehrlenmeyer flasks, beakers, or cylinders. The surgical instrument sets are often grouped together and sterilized, wrapped or sealed, stored in a container, and finally transported to an operating room for use. Increasingly, standards widely adopted in the medical industry dictate particular tray or container sizes in an effort to provide uniformity and compatibility between different sterilization systems, storage systems, and other related equipment. One such standard is set forth by the Deutsches Institut für Normung (DIN), and another recognized standard for sterilization tray sizes or containers is set forth by the International Organization for Standardization (ISO).

Complex procedures may involve a substantial number of instruments and accordingly, several instrument trays may be necessary to accommodate all of the required surgical instruments. Moreover, depending on the tools and instruments employed, there may be considerable unused space in one or more of the standard-sized trays. Not only is space in an operating room often limited, but seeking instruments that may be inefficiently scattered throughout a number of standard-sized trays can result in loss of precious time during a procedure. In extreme situations, time lost could compromise the safety of a patient in addition to wasteful utilization of operating room personnel in handling or searching through trays to access certain instruments.

Efforts to address these shortcomings have been undertaken. For example, in some cases, fixation modules are provided to segregate and fix the positions of the instruments. Invariably, however, there is limited flexibility in the placement of those modules within the container. Other attempts may present variations in a container that take the container outside of the adopted standards (e.g., DIN or ISO) in a particular health services organization, thus rendering them incompatible with storage systems, sterilizations systems or the like already in place. Even further, trays are typically wrapped in paper or cloth to maintain the sterility of the contents in storage. Efforts to provide modular containers or trays often include one or more protruding fasteners or edges that can catch and rip the sterilized packaging of an instrument set before reaching a sterile field—rendering it unusable until re-sterilized.

In view of the above, it is desirable to provide a modular storage or delivery system for surgical instruments that addresses and overcomes these shortcomings.

### SUMMARY OF THE INVENTION

The present invention advantageously provides modular container systems for medical instrument storage and/or delivery that provide increased space efficiency and customization, while remaining compatible with existing systems or devices adopting a standard size or criteria for compatibility. For example, a modular container system for sterilized medical instruments is provided, including a first container including a first base, a first plurality of sidewalls, and a first lid; a second container including a second base, a second plurality of sidewalls, and a second lid; and an interlocking element that releasably engages at least one of the first plurality of sidewalls with at least one of the second plurality of sidewalls, where the interlocking element defines an exterior surface substantially coplanar with the at least one of the first plurality of sidewalls and the at least one of the second plurality of sidewalls when engaged. The first container defines a first height, a first length, and a first width, and the second container defines a second height, a second length, and a second width, where the first height is substantially equal to the second height, the first width is substantially equal to the second width, and the first length is approximately one-half of the second length. A size of the interlocked first and second containers may conform to an industry standard size such as DIN or ISO standards. The first lid may be pivotable with respect to at least one of the first plurality of sidewalls about a first joint, where the first joint defines an exterior surface substantially coplanar with the first lid. The second lid may be pivotable with respect to at least one of the second plurality of sidewalls about a second joint, where the second joint defines an exterior surface substantially coplanar with the second lid. The system may include a handle movably attached to at least one of the first or second containers, where the handle is positionable into a substantially coplanar position with at least one of the first or second plurality of sidewalls.

A modular medical container system is provided, including a first container including a first base, a first plurality of sidewalls, and a first lid; a second container including a second base, a second plurality of sidewalls, and a second lid; and a first interlocking element that releasably engages at least one of the first plurality of sidewalls with at least one of the second plurality of sidewalls, such that, when engaged, the first and second containers define a substantially smooth exterior profile. A size of the exterior profile may conform to an industry standard size. The second container may define a length that is approximately twice a length defined by the first container. When engaged, the first lid may define a substantially continuous, planar surface with the second lid and/or the first base may define a substantially continuous, planar surface with the second base. At least one of the first plurality of sidewalls and at least one of the second plurality of sidewalls may be substantially coplanar. The first interlocking element may define an exterior surface substantially coplanar with the at least one of the first plurality of sidewalls and the at least one of the second plurality of sidewalls when engaged. The at least one of the first plurality of sidewalls may define a recessed cavity, and the at least one of the second plurality of sidewalls may define a protruding portion positionable within the cavity to facilitate releasable engagement between the first and second containers. The system may include a handle movably attached to at least one of the first or second containers, where the handle is positionable into a substantially coplanar position with at least one of the first or second plurality of sidewalls.

A method of packaging sterilized medical equipment is provided, including providing a plurality of first containers each having a first size; placing at least one sterilized medical device in a first container of the plurality; providing a plurality of second containers each having a second size different from the first size; placing at least one sterilized medical device in a second container of the plurality; and releasably engaging the first container laterally to the second container such that the engaged first and second containers define a substantially smooth exterior profile, where a size of the exterior profile conforms to an industry standard size. At least one sterilized medical device in the first container may be wrapped in a sterile dressing, and/or at least one sterilized medical device in the second container may be wrapped in a sterile dressing. The second container may define an interior volume approximately twice an interior volume defined by the first container.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of an example of a modular sterilization delivery system constructed in accordance with the principles of the present invention;

FIG. 2 is an illustration of an example of an attachment mechanism for the modular sterilization delivery system of FIG. 1; and

FIG. 3 is another illustration of an example of an attachment mechanism for the modular sterilization delivery system of FIG. 1.

### DETAILED DESCRIPTION OF THE INVENTION

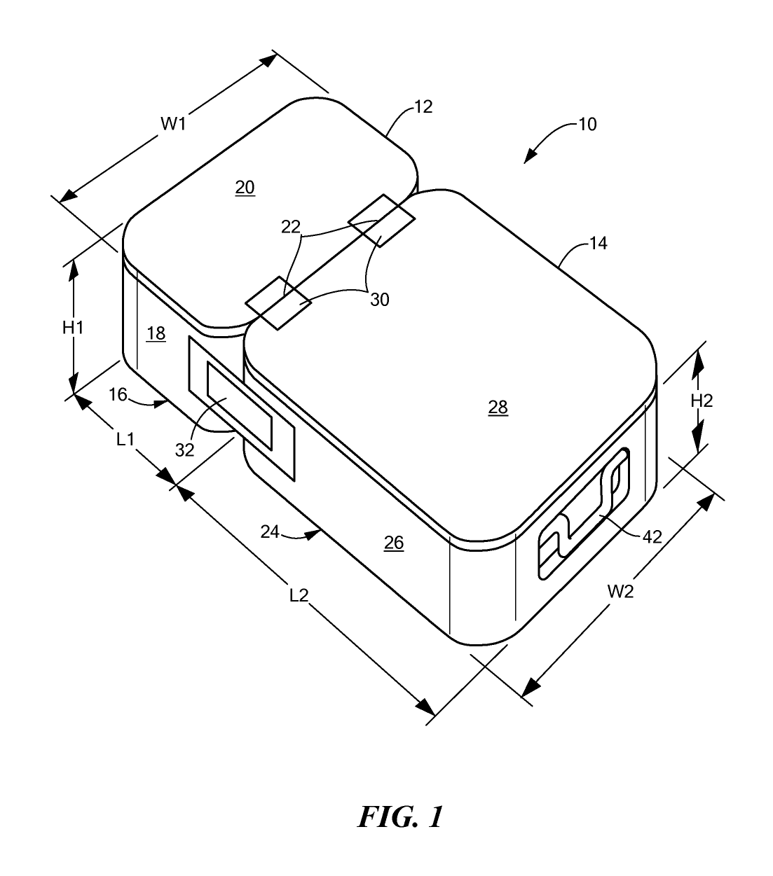

The present invention advantageously provides modular container systems for medical instrument storage and/or delivery that provide increased space efficiency and customization, while remaining compatible with existing systems or devices adopting a standard size or criteria for compatibility. Now referring to the figures in which like reference designators refer to like elements, there is shown in FIGS. 1-3 an exemplary modular container system for sterilized medical devices constructed in accordance with the principles of the present invention, designated generally as “10.” The system 10 generally provides a plurality of releasably interlocking containers that, when coupled to one another, conform to an industry standard size, such as DIN or ISO standards for example. In addition, the system 10 presents interlocked or engaged containers presenting a substantially smooth exterior profile that reduces the likelihood of snagging or tearing sterilization wraps, and further allowing the containers to be readily stacked or slidably inserted into exiting storage systems, shelves, sterilization devices, or the like conforming to the adopted standard.

In particular, the system 10 may include a first container 12 releasably engageable with a second container 14. The first container 12 may include or define a first base 16 and a plurality of sidewalls 18 coupled to the base to define an interior cavity or chamber therein for receiving and storing sterilized medical instruments. The first container 12 may also include a first lid 20 movably coupled to the sidewalls to allow or obstruct access to the interior cavity or chamber. The first lid 20 may be pivotable with respect to at least one of the first plurality of sidewalls 18 about one or more joints 22, where the joint(s) 22 defines an exterior surface substantially coplanar with the first lid 20. The lid 20 thus presents a smooth upper surface free of protrusions that could interfere with stacking or sliding the tray, while also eliminating potential tearing surfaces. The first container 12 may define a first height H1, a first width W1, and a first length L1 resulting in a first storage volume therein.

The second container 14 may include or define a second base 24 and a plurality of sidewalls 26 coupled to the base 24 to define an interior cavity or chamber therein for receiving and storing sterilized medical instruments. The second container 14 may also include a second lid 28 movably coupled to the sidewalls to allow or obstruct access to the interior cavity or chamber. The second lid 28 may be pivotable with respect to at least one of the plurality of sidewalls about one or more joints 30, where the joint(s) defines an exterior surface substantially coplanar with the second lid 28. In addition, when the first container 12 is interlocked, the first and second lids may define a substantially continuous, planar surface facilitating stacking, sliding. The first and second bases may similarly define a substantially continuous, planar surface when the first and second containers are attached to one another. The second container 14 may define a second height H2, a second width W2, and a second length L2 resulting in a second storage volume therein. The size of the second container 14 may differ from the size of the first container 12. For example, the second length of the second container 14 may be twice the size of the first length of the first container 12, resulting in twice the interior volume.

The releasable engagement between the first and second containers may be effected while maintain or presenting a substantially smooth exterior profile or shape to prevent or minimize sharp edges or protruding surfaces that could snag sterile wrapping or dressing surrounding an instrument or tool being inserted into or removed from either of the containers. For example, the system 10 may include a first interlocking element or mechanism 32 that engages at least one of the sidewalls of the first container 12 to at least one of the sidewalls of the second container 14. The first interlocking element 32 may, for example, extend along or engage a length of two exterior sidewalls that are substantially parallel to one another, as shown in FIG. 1. The first interlocking element 32 may include a latching mechanism presenting a substantially smooth exterior surface that is substantially parallel to the sidewalls it engages, thus presenting a smooth, snag-free profile.

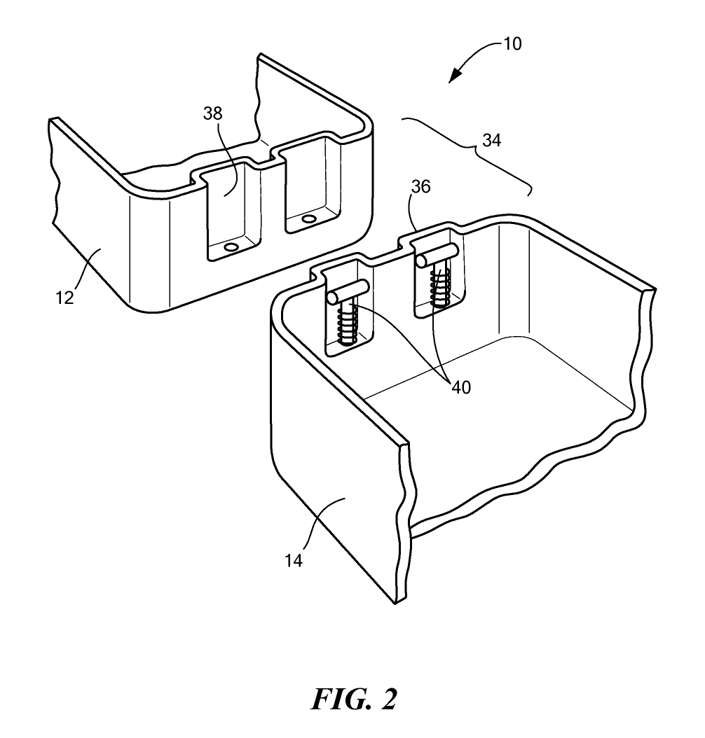

Now referring to FIG. 2, the system 10 may include a second interlocking element or mechanism 34 that engages two oppositely-facing sidewalls of the first and second containers, respectively. The second interlocking element or mechanism 34 may include, for example, one or more protruding portions 36 on a sidewall of the second container 14 that matably couple or are positionable within one or more depressions or recessed cavities 38 in a sidewall of the first container 12. The second interlocking element 34 may also include one or more pins or rods 40 that releasably secure the protruding portions 36 to the recessed cavities 38. The rods or pins 40 may be spring mounted to automatically return to a locked positioned when the two containers are aligned.

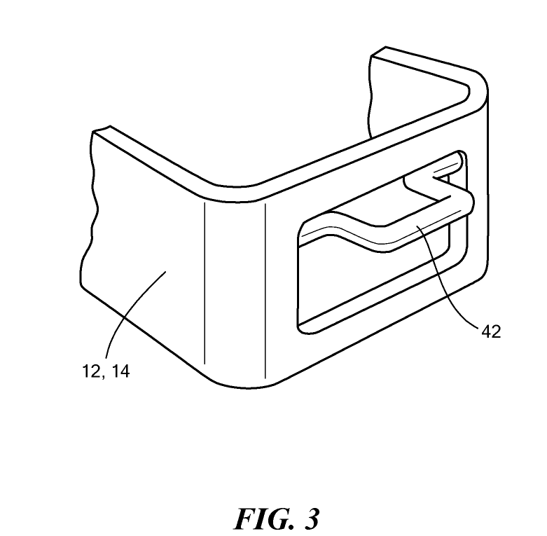

Now referring to FIG. 3, the first and/or second container 14 may include a handle 42 on a sidewall thereof to facilitate carrying or manipulating the container(s). The handle 42 may be movable with respect to the first and/or second container 14 from a first position where the handle 42 is outwardly facing and readily graspable by a user (as shown in FIG. 3), to a second position where the handle 42 is substantial parallel to the sidewall of the first and/or second container 14 (as shown in FIG. 1). When in the second position, the handle 42 does not significantly protrude from a plane defined by the sidewall, and thus again reduces or eliminates a potential pinch point or snagging surface that could lead to unintentional tearing of a sterile wrap or dressing.

The size of the interlocked first and second containers may conform to an industry standard size, such as a DIN or ISO standard. The modularity of the system 10 combined with its compatible sizing when connected allows the present system 10 to be readily integrated and used with existing storage, sterilization, or other infrastructure conforming to the standard and adapted for us in conjunction with storage or sterilization trays. Moreover, increased efficiency and customization can be achieved not only on-site in an operating room or medical service provider location, but also at a distributor or customer service level. For example, a plurality of the first containers may be stored or housed at a distribution location, where the first containers each contain one or more variations of sets or subsets of medical instruments or devices ready for use. The instruments or devices may be sterilized, wrapped in a sterile dressing, and securely sealed or otherwise placed in the first container(s). In addition, a plurality of the second containers may be stored or housed at the distribution location, where the second containers each contain one or more varying sets or subsets of medical instruments or devices for use. When a medical service provide requests or orders a surgical instrument set for a designated procedure, the requisite tools in the pre-arranged, varying plurality of first and second containers can simply be selected, interlocked, and delivered to the service provider, rather than having to individually assemble full-length, standardized trays in an effort to maximize space while satisfying the service providers requirements. This results in a drastically reduced time required to fulfill customer orders, and likely reduces the overall space or number of trays needed to accommodate a particular instrument set.

It will be appreciated by persons skilled in the art that the present invention is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the invention. A variety of modifications and variations are possible in light of the above teachings without departing from the scope and spirit of the invention, which is limited only by the following claims.

What is claimed is:

1. A modular container system for sterilized medical instruments, comprising:
a first container including a first base, a first plurality of sidewalls, and a first lid;
a second container including a second base, a second plurality of sidewalls, and a second lid; and
an interlocking element that releasably engages at least one of the first plurality of sidewalls with at least one of the second plurality of sidewalls, wherein the interlocking element defines an exterior surface substantially coplanar with the at least one of the first plurality of sidewalls and the at least one of the second plurality of sidewalls when engaged.

2. The system of claim 1, wherein the first container defines a first height, a first length, and a first width, and the second container defines a second height, a second length, and a second width, wherein the first height is substantially equal to the second height, the first width is substantially equal to the second width, and the first length is approximately one-half of the second length.

3. The system of claim 2, wherein a size of the interlocked first and second containers conforms to an industry standard size.

4. The system of claim 1, wherein the first lid is pivotable with respect to at least one of the first plurality of sidewalls about a first joint, wherein the first joint defines an exterior surface substantially coplanar with the first lid.

5. The system of claim 4, wherein the second lid is pivotable with respect to at least one of the second plurality of sidewalls about a second joint, wherein the second joint defines an exterior surface substantially coplanar with the second lid.

6. The system of claim 1, further comprising a handle movably attached to at least one of the first or second containers, wherein the handle is positionable into a substantially coplanar position with at least one of the first or second plurality of sidewalls.

7. A modular medical container system, comprising:
a first container including a first base, a first plurality of sidewalls, and a first lid;
a second container including a second base, a second plurality of sidewalls, and a second lid; and
a first interlocking element that releasably engages at least one of the first plurality of sidewalls with at least one of the second plurality of sidewalls, such that, when engaged, the first and second containers define a substantially smooth exterior profile.

8. The system of claim 7, wherein a size of the exterior profile conforms to an industry standard size.

9. The system of claim 8, wherein the second container defines a length that is approximately twice a length defined by the first container.

10. The system of claim 7, wherein when engaged, the first lid defines a substantially continuous, planar surface with the second lid.

11. The system of claim 7, wherein when engaged, the first base defines a substantially continuous, planar surface with the second base

12. The system of claim 7, wherein the at least one of the first plurality of sidewalls and the at least one of the second plurality of sidewalls are substantially coplanar.

13. The system of claim 12, wherein the first interlocking element defines an exterior surface substantially coplanar with the at least one of the first plurality of sidewalls and the at least one of the second plurality of sidewalls when engaged.

14. The system of claim 7, wherein the at least one of the first plurality of sidewalls defines a recessed cavity, and the at least one of the second plurality of sidewalls defines a protruding portion positionable within the cavity.

15. The system of claim 7, further comprising a handle movably attached to at least one of the first or second containers, wherein the handle is positionable into a substantially coplanar position with at least one of the first or second plurality of sidewalls.

16. A method of packaging sterilized medical equipment, comprising:
providing a plurality of first containers each having a first size;
placing at least one sterilized medical device in a first container of the plurality;
providing a plurality of second containers each having a second size different from the first size;
placing at least one sterilized medical device in a second container of the plurality; and
releasably engaging the first container laterally to the second container such that the engaged first and second containers define a substantially smooth exterior profile, wherein a size of the exterior profile conforms to an industry standard size.

17. The method of claim 16, wherein the at least one sterilized medical device in the first container is wrapped in a sterile dressing.

18. The method of claim 16, wherein the at least one sterilized medical device in the second container is wrapped in a sterile dressing.

19. The method of claim 16, wherein the second container defines an interior volume approximately twice an interior volume defined by the first container.
